(19) United States
(12) Patent Application Publication
(10) Pub. No.: US 2023/0174949 A1
(43) Pub. Date: Jun. 8, 2023

(54) ENGINEERED VASCULARIZED ORGANOIDS

(71) Applicant: The Regents of the University of California, Oakland, CA (US)

(72) Inventors: Prashant Mali, La Jolla, CA (US); Amir Dailamy, La Jolla, CA (US); Udit Parekh, La Jolla, CA (US)

(21) Appl. No.: 18/074,402

(22) Filed: Dec. 2, 2022

Related U.S. Application Data
(60) Provisional application No. 63/285,911, filed on Dec. 3, 2021.

(51) Int. Cl. C12N 5/074; C12N 15/85

(52) U.S. Cl. CPC C12N 5/0696; C12N 15/85; C12N 2533/90; C12N 2501/60; C12N 2513/00

(57) ABSTRACT

Provided are methods and compositions for tissue engineering including methods and compositions for the generation of vascularized organoids in vitro.

### CROSS REFERENCE TO RELATED APPLICATIONS

This application claims priority under 35 U.S.C. § 119 from Provisional Application Ser. No. 63/285,911, filed Dec. 3, 2021, the disclosures of which are incorporated herein by reference in its entirety.

### STATEMENT REGARDING FEDERALLY SPONSORED RESEARCH

This invention was made with Government support under RO1HG009285, RO1CA222826, and RO1GM123313 awarded by the National Institutes of Health. The government has certain rights in the invention.

### TECHNICAL FIELD

Provided are methods and compositions for tissue engineering including methods and compositions for the generation of vascularized organoids in vitro.

### BACKGROUND

Pluripotent stem cell-derived organoids have transformed the ability to recreate complex three-dimensional models of human tissue. However, the directed differentiation methods used to create them do not afford the ability to introduce cross-germ-layer cell types.

The ability to recapitulate organogenesis and create complex human tissue in vitro has been a long-standing goal for the stem cell and tissue engineering field. The advent of organoid technology has recently made it possible to create three-dimensional (3D), self-organized, pluripotent stem cell (PSC)-derived tissues for in vitro developmental and disease modeling that closely mimic the cellular, spatial, and molecular architecture of endogenous human tissue (Clevers, 2016; Lancaster and Knoblich, 2014). These advances have enabled substantial progress in building fully PSC-derived, functional human organs in vitro (Takebe and Wells, 2019). However, the absence of vasculature in most organoid techniques limits their utility, principally in two ways. Firstly, it is widely accepted that vasculature plays a crucial role in development and disease (Daniel and Cleaver, 2019; Petrova and Koh, 2018). Secondly, vasculature is necessary to prevent necrosis in tissues that grow beyond 1 mm in size (Grebenyuk and Ranga, 2019; Lancaster, 2018), which deems vasculature critical for building large-scale tissue models.

To address this, several groups have demonstrated progress on developing methods for vascularizing organoids (Cakir et al., 2019; Garreta et al., 2019; Guye et al., 2016; Homan et al., 2019; Low et al., 2019; Mansour et al., 2018; Pham et al., 2018). Some groups have succeeded in vascularizing organoids after transplanting them in vivo (Mansour et al., 2018; Pham et al., 2018), but requiring an in vivo host limits experimental control, increases cost, and diminishes its potential for clinical applications. Others transiently overexpressed GATA6 to introduce a nascent vascular network in liver-bud organoids (Guye et al., 2016); however, there is no current evidence this system can be translated to other organoids.

Organoid platforms leverage knowledge of development to provide temporally appropriate chemical cues to self-assembled PSC-derived embryoid bodies, modulating key organogenesis-specific signaling pathways to drive the directed differentiation of organ-specific cells in physiologically faithful architectures. Thus, cells that do not belong to those specific organ compartments or arise from different germ layers are absent from the final organoid. To introduce cell types outside of those available from directed differentiation, a promising strategy is to combine genetic overexpression with directed differentiation. Recently, vascularization of cerebral organoids has been reported, in which PSCs were engineered to ectopically express human ETS variant 2 (ETV2) (Cakir et al., 2019), a known driver of differentiation to endothelial cells from PSCs (Lindgren et al., 2015; Parekh et al., 2018). Although this method has exciting potential, it suffers from two limitations: one, it induces only a low degree of vascularization, and, two, it does not induce the full panoply of vascular lineages, such as smooth muscle cells (SMCs) and mesenchymal stem cells (MSCs), which are critical for blood vessel development and function (Ferland-McCollough et al., 2017). Thus, there is a need to explore alternative methods.

A recently described vascular organoid (VO) differentiation approach (Wimmer et al., 2019) yields complete blood vessel networks, including SMCs, MSCs, and endothelial cells, but these organoids lack organ-specific parenchymal cells, limiting their utility for broader disease modeling and regenerative medicine.

### SUMMARY

The disclosure provides a bottom-up engineering approach to building vascularized human tissue by combining genetic reprogramming with chemically directed organoid differentiation. As a proof of concept, the disclosure demonstrates this approached in neuro-vascular and myo-vascular organoids via transcription factor overexpression in vascular organoids. A comprehensive characterization of neuro-vascular organoids was performed in terms of marker gene expression and composition, and the results demonstrated that the organoids maintain neural and vascular function in culture. The exemplary organoid also showed chronic electrical stimulation of myo-vascular organoid aggregates as a potential path toward engineering mature and large-scale vascularized skeletal muscle tissue from organoids. The approach offers a roadmap to build diverse vascularized tissues of any type derived entirely from pluripotent stem cells.

The disclosure provides a method of reprogramming with the directed differentiation of VOs to derive cross-germ-layer and cross-lineage organoids with complete vascular networks. a parenchymal cell component is introduced into VOs via transcription factor (TF) overexpression and demonstrate this approach by building neuro-vascular and myo-vascular organoids. This is done by the induced overexpression, in developing vascular organoids, of NEUROD1 (iN) to form neuro-vascular organoids (iN-VOs), and the induced overexpression of MYOD1 plus BAF60C (iMB) to form myo-vascular organoids (iMB-VOs).

This yields a facile method for co-differentiation of tissue-specific parenchymal cells and the entire blood vessel lineage from a single PSC line. This approach is presented as a proof of concept for the introduction of other parenchymal cell types, via the overexpression of lineage-specifying TFs, in the context of a VO scaffold.

The disclosure demonstrates that a combination of directed differentiation and the overexpression of a lineage-specific reprogramming factor can build cross-lineage and cross-germ-layer organoid systems. To exemplify the approach the disclosure introduced neurons and skeletal muscle into VOs, as a proof of principle of such an approach. These mixed-lineage organoids were then assayed to demonstrate that this combined approach yielded neurons only upon induction of NEUROD1 overexpression, and skeletal muscle only upon induction of MYOD1+BAF60C overexpression, while retaining the architecture of the VO. The neuro-vascular organoids thus generated were further optimized for long-term culture, and comprehensively characterized for composition and to confirm function of both lineage branches. Finally, for the myo-vascular organoids, the maturation of these organoids was demonstrated via chronic electrical stimulation, which enhanced the expression of skeletal muscle myosins and calcium-handling genes.

The disclosure provides a blueprint for complete bottom-up engineering of vascularized organoids, where genetic perturbation methods for cell reprogramming can be overlaid onto a core protocol to introduce additional cell types. This framework is a powerful platform to generate diverse organoids, thus laying the foundation for completely in vitro-derived, large-scale tissue systems for regenerative medicine purposes.

A method of producing vascularized tissue in vitro comprising transforming a first population of pluripotent stem cell with a vector encoding one or more transcription factors that induce differentiation of the first population of pluripotent stem cell down a particular lineage path under the control of an inducible promoter to obtain parenchymal-inducible stem cells; combining the parenchymal-inducible stem cells with a second population of pluripotent stem cells lacking the vector under suitable co-culture conditions; and inducing the second population of pluripotent stem cells down a vascular committed lineage pathway and either before, during or after inducing vascularization inducing expression of the one or more transcription factors in the parenchymal-inducible stem cells to cause the parenchymal-inducible stem cells to become parenchymal cells, wherein a vascularized organoid tissue is obtain. In one embodiment, the pluripotent stem cells are selected from embryonic stem cells, pluripotent stem cells, and induced pluripotent stem cells. In another embodiment, the vector is a piggybac vector. In still another embodiment, the organoid tissue is a neuro-vascular tissue. In another embodiment, the organoid tissue is a myo-vascular tissue. In yet another embodiment, the parenchymal cell is selected from the group consisting of astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, and/or epididymal basal cells. In another embodiment, the first population of pluripotent stem cells are layered in or on a first biocompatible matrix and the second population of pluripotent stem cells are layered in or on a second biocompatible matrix. In a further embodiment, the first and second biocompatible matrix comprise the same material. In yet another further embodiment, the first and second biocompatible matrix comprise different material compositions. In still another embodiment, the biocompatible matrix comprises a material selected from the group consisting of nylon (polyamides), dacron (polyesters), polystyrene, polypropylene, polyacrylates, polyvinyl compounds, polycarbonate (PVC), polytetrafluorethylene (PTFE, teflon), thermanox (TPX), nitrocellulose, cotton, polyglycolic acid (PGA), collagen, cat gut sutures, cellulose, and gelatin. In another embodiment, the first and/or second biocompatible matrix are 3 dimensional porous materials. In one embodiment, parenchymal-inducible stem cells are expanded in culture prior to combining with the second population of pluripotent stem cells.

The disclosure also provides a method of generating a vascular organoids comprising seeding a stem cell population comprising a vector containing one or more lineage committed transcription factors operably linked to an inducible promoter onto a vascularized organoid scaffold. In a further embodiment, the vascularized organoid scaffold is generated from stem cells.

The disclosure further provides an engineered vascular tissue produced by the method of the disclosure, wherein the tissue comprises cells having an inducible promoter operably linked to a transcription factor.

### DESCRIPTION OF DRAWINGS

FIG. 1A-E provides construction and characterization of inducible cell lines. (A) Schematic of PiggyBac transposon-based inducible overexpression vector. (B) Schematic of cell line generation and validation process. (C) Inducible NEUROD1 (iN) cell line validation at 3 weeks post induction via (1) qRT-PCR analysis of signature neuronal markers MAP2, TUBB3, VGLUT2, and VGAT; data represent the mean±SD (n=4 independent experiments); (2) immunofluorescence micrograph of MAP2+ cells (scale bars, 50 mm); and (3) representative spike plots from MEA measurements of spontaneously firing iN cells. (D) Inducible ASCL1+DLX2 (iAD) cell line validation at 3 weeks post induction via (1) qRT-PCR analysis of signature neuronal markers MAP2, TUBB3, VGLUT2, and VGAT; data represent the mean±SD (n=4 independent experiments); (2) immunofluorescence micrograph of MAP2+ cells (scale bars, 50 mm); and (3) representative spike plots from MEA measurements of spontaneously firing iAD cells. (E) Inducible MYOD1+BAF60C (iMB) cell line validation at 2 weeks post induction via (1) qRT-PCR analysis of signature skeletal muscle markers MYH8, TNNC1, and RYR; data represent the mean±SD (n=3 independent experiments); and (2) immunofluorescence micrograph of MYH+-, MYOG+-, and SAA+-labeled cells (scale bars, 50 mm). (C-E) **p %0.01, ***p %0.001, and ****p %0.0001; ns, not significant.

FIG. 2A-H provides generation of iN-VOs and iMB-Vos. (A) General strategy for the generation of vascularized organ tissues via introduction of parenchymal cell types in VOs. (B) Schematic of iN-VO and iMB-VO culture protocol. (C) Immunofluorescence 100-μm z stack, maximum projection, confocal micrographs of MAP2- and CDH5-labeled uninduced (iN-VO, -Dox) and induced (iN-VO, +Dox) day 15 iN-VO organoids (scale bars, 50 μm). (D) qRT-PCR analysis of signature endothelial genes CDH5 and VEPTP; signature smooth muscle gene SMA; and signature neuronal genes MAP2, VGLUT2, BRN2, and FOXG1 at day 15 of culture for iN-VO organoids. Data represent the mean±SD (n=7 organoids, from three independent experiments). (E) Immunofluorescence 100-μm z stack, maximum projection, confocal micrographs of MYH- and CDH5-labeled uninduced (iMB-VO, -Dox) and induced (iMB-VO, +Dox) day 15 iMB-VO organoids (scale bars, 50 μm). (F) qRT-PCR analysis of signature endothelial genes CDH5 and VEPTP; signature smooth muscle gene SMA; and signature skeletal muscle genes MYOG, MYH8, TNNC1, and RYR at day 15 of culture for iMB-VO organoids. Data represent the mean±SD (n=7 organoids, from three independent experiments). (G) Pan-organoid tile-scan immunofluorescence confocal micrograph of a CDH5- and MAP2-labeled day 15 neuro-vascular organoid (scale bars, 500 μm). Image is a 200-μm z stack maximum intensity projection. (H) Pan-organoid tile-scan immunofluorescence confocal micrograph of CDH5- and MYH-labeled day 15 myo-vascular organoid (scale bars, 500 μm). Image is a 200-μm z stack maximum intensity projection. (D and F) **p≤0.01, ***p≤0.001, and ****p≤0.0001; ns, not significant.

FIG. 3A-G shows Molecular and functional characterization of iN-VOs. (A) Outline of long-term cultured iN-VO characterization. (B) qRT-PCR analysis of signature endothelial genes CDH5 and VEPTP, signature smooth muscle gene SMA, and signature neuronal genes MAP2 and BRN2 at day 30 of iN-VO culture. Data represent the mean±SD (n=7 organoids, from three independent experiments). (C) Immunofluorescence 100-μm z stack, maximum projection, confocal micrographs of MAP2+- and CDH5+-induced day 30 iN-VOs (scale bars, 100 μm). (D) Immunofluorescence 100-μm z stack, maximum projection, confocal micrographs of PDGFR+- and CDH5+-induced day 30 iN-VOs (scale bars, 50 μm). (E) Uniform manifold approximation and projection (UMAP) visualization of cell types from day 45 iN-VOs. Two independent induction conditions, along with one non-induction condition. (F) Cluster-specific expression of marker genes in day 45 iN-VOs. (G) Experimental validation of iN-VO perfusibility in vivo by subcutaneous implantation of iN-VO, showing immunofluorescence micrographs of intravital Dextran, CDH5, and overlay (scale bar, 25 μm). Representative image from two independent experiments.

FIG. 4A-E provides Electrical characterization and stimulation of organoids. (A) Schematic of iN-VO electrical characterization via MEA recordings. (B) Image of day 30 iN-VOs plated on microelectrode array (scale bars, 500 μm). (C) Representative spike plots from MEA measurements of spontaneously firing iN-Vos and corresponding raster plot. Representative image and plot from two independent experiments. (D) Schematic of iMB-VO in vitro maturation by electrical stimulation. (E) qRT-PCR analysis of skeletal muscle myosins: MYH2, MYH3, MYH7, and MYH8, and genes involved in calcium handling, CASQ1, CASQ2, SERCA1, SERCA2, and RYR for stimulated versus unstimulated iMB-VOs. Data represent the mean±SD (n=6 organoids, from two independent experiments). **p≤0.01, ***p≤0.001, and ****p≤0.0001; ns, not significant.

FIG. 5A-F shows Optimization of organoid culture conditions. (A) Schematic for experiment to assess the optimal ratio of iN to WT cells for iN-VO formation. (B) qRT-PCR analysis of signature neuronal gene MAP2, and signature endothelial gene CDH5, at day 15 of culture for organoids grown from 50% and 100% iN cells. Data represent the mean±s.d. (n=3 organoids) and the unpaired two-tailed t-test was used for all comparisons. (C) Side profile 3D rendering of a panorganoid tile-scan, z-stack immunofluorescence confocal micrograph of a CDH5- and MAP2-labelled day 15 neuro-vascular organoid (Scale bars=150 μm). (D) Side profile 3D rendering of a pan-organoid tile-scan, z-stack immunofluorescence confocal micrograph of CDH5- and MYH labelled day 15 myo-vascular organoid (Scale bars=150 μm). (E) Schematic for experiment to assess optimal media supplements for long term iN-VO culture. (F) qRT-PCR analysis of signature neuronal gene MAP2, and signature endothelial gene CDH5, at day 30 of culture to assess long term neuronal and endothelial survival. Data represent the mean±s.d. (n=3 organoids) and the unpaired two-tailed t-test was used for all comparisons. (b,f) Statistical significance was attributed to P<0.05 as determined by unpaired two-tailed t-test comparison. (**P≤0.01, ***P≤0.001, and ****P≤0.0001; ns=not significant.

FIG. 6A-E shows scRNA-seq characterization of neurovascular organoids. (A) UMAP visualization of iN-VO clusters annotated by sample type. Two independent induction conditions, along with one non-induction condition. (B) Expression of marker genes for each classified cell type. (C) Mapping of neurovascular organoid cell types to cell types in the mouse cell atlas (Han et al. 2018). (D) UMAP visualization of clusters from a reference dataset of cells profiled during 2D differentiation of neurons from pluripotent stem cells by NGN2 overexpression (Schornig et al. 2021). Cells were profiled at day 14 and day 35 after induction. Neuronal clusters in the 2D differentiated neurons are highlighted below by overlaying DCX expression on the UMAP visualization. (E) Mapping of neurovascular organoid neurons to clusters in the 2D differentiated neurons.

FIG. 7A-D shows Extensive molecular and phenotypic characterization of long-term cultured iNVOs. (A) The left image shows orthogonal sections of confocal stacks from neuro-vascular organoids stained for CDH5 (red) and Col IV (Scale bars=50 μm). The right image shows a zoomed in view of formed lumens, as indicated with arrowheads (Scale bars=25 μm). (B) Immunofluorescence 100 μm z-stack, maximum projection, confocal micrographs of Day 30 neuro-vascular organoids labelled for CDH5 post Dil-ac-LDL endothelial uptake assay (Scale bars=50 μm). (C) Further characterization of endothelial functionality in vivo via intravital perfusion shows: (i) Experimental validation of pericyte recruitment of in vivo perfused neuro-vascular organoids, showing immunofluorescence micrograph of intravital Dextran and PDGFRB overlay (Scale bar=50 μm); (ii) 100 μm z-stack projection that spans a 650×650 μm tilescan region of whole-mount neuro-vascular organoid implant showing intravital Dextran and CDH5 overlay (Scale bar=100 μm); and (iii) Orthogonal sections of confocal stacks from neuro-vascular organoids implants 90 days post-implantation, stained for CDH5 (green) and showing dextran perfused within the vessel. Lumens are indicated with arrowheads (Scale bars=20 μm). (D) Wildtype mouse kidney stained for human-specific anti-CDH5 and anti-SMA antibodies. Note the absence of the CDH5 signal (Scale bar=10 μm).

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the fragment” includes reference to one or more fragments and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also can refer to both double and single stranded molecules. Unless otherwise specified or required, any embodiment of this disclosure that is a polynucleotide can encompass both the double stranded form and each of two complementary single stranded forms known or predicted to make up the double stranded form.

Embryonic stem cells (ESCs) are derived from the inner cell mass (ICM) of a blastocyst. ESCs can grow infinitely (self-renewal) while maintaining the ability to generate all somatic cell types and germ cell lineages (pluripotency). This specific stem cell function has been investigated at the molecular level, leading to the identification of the key roles of master transcription factors such as Oct3/4 and Nanog, and secreted signaling molecules including leukemia inhibitory factor (LIF), bone morphogenetic proteins (BMP) and Wnt. Recent derivation of hESCs has opened the door to the use of pluripotent stem cells as a source of the cell transplantation therapy for the treatment of diseases such as Parkinson's disease and diabetes mellitus. With exponentially increasing demand for hESCs research toward medical applications, it is essential to investigate how to regulate pluripotency in order to develop uncompromised technologies to derive and expand hESCs that is at the stem of entire cell therapeutic strategies before inducing any differentiated type of adult cells.

Embryonic stem cells are generated and maintained using methods well known to the skilled artisan such as those described by Doetschman et al. (1985) J. Embryol. Exp. Mol. Biol. 87:27-45). Any line of ES cells can be used. One mouse strain that is typically used for production of ES cells, is the 129J strain. Another ES cell line is murine cell line D3 (American Type Culture Collection, catalog no. CKL 1934). Still another ES cell line is the WW6 cell line (Ioffe et al. (1995) PNAS 92:7357-7361). Human embryonic stem cells (hESCs) can be isolated, for example, from human blastocysts obtained from human in vivo preimplantation embryos, in vitro fertilized embryos, or one-cell human embryos expanded to the blastocyst stage (Bongso, et al. (1989), Hum. Reprod., vol. 4: 706). Human embryos can be cultured to the blastocyst stage in G1.2 and G2.2 medium (Gardner, et al. (1998), Fertil. Steril., vol. 69:84). The zona pellucida is removed from blastocysts by brief exposure to pronase (Sigma). The inner cell masses can be isolated by immunosurgery or by mechanical separation, and are plated on mouse embryonic feeder layers, or in the defined culture system as described herein. After nine to fifteen days, inner cell mass-derived outgrowths are dissociated into clumps either by exposure to calcium and magnesium-free phosphate-buffered saline (PBS) with 1 mM EDTA, by exposure to dispase, collagenase, or trypsin, or by mechanical dissociation with a micropipette. The dissociated cells are then replated as before in fresh medium and observed for colony formation. Colonies demonstrating undifferentiated morphology are individually selected by micropipette, mechanically dissociated into clumps, and replated. Embryonic stem cell-like morphology is characterized as compact colonies with apparently high nucleus to cytoplasm ratio and prominent nucleoli. Resulting embryonic stem cells are then routinely split every 1-2 weeks by brief trypsinization, exposure to Dulbecco's PBS (without calcium or magnesium and with 2 mM EDTA), exposure to type IV collagenase (about 200 U/mL), or by selection of individual colonies by mechanical dissociation, for example, using a micropipette.

Once isolated, the stem cells, can be cultured in a culture medium that supports the substantially undifferentiated growth of stem cells using any suitable cell culturing technique. For example, a matrix laid down prior to lysis of primate feeder cells (preferably allogeneic feeder cells) or a synthetic or purified matrix can be prepared using standard methods. The stem cells to be cultured are then added atop the matrix along with the culture medium. In other embodiments, once isolated, undifferentiated stem cells can be directly added to an extracellular matrix that contains laminin or a growth-arrested human feeder cell layer (e.g., a human foreskin fibroblast cell layer) and maintained in a serum-free growth environment. In yet another embodiment, the stem cells can be directly added to a biocompatible cell culture plate in the absence of an extracellular matrix material (e.g., directly on polystrene, glass or the like). Unlike existing embryonic stem cell lines cultured using conventional techniques, embryonic stem cells and their derivatives prepared and cultured as described herein avoid or have reduced exposure to xenogeneic antigens that may be present in feeder layers. This is due in part to the media compositions promoting growth in the absence of feeder layers or directly on a cell culture substrate. This avoids the risks of contaminating human cells, for example, with non-human animal cells, transmitting pathogens from non-human animal cells to human cells, forming heterogeneous fusion cells, and exposing human cells to toxic xenogeneic factors.

In yet another aspect, mesenchymal stem cells can be used. Mesenchymal stem cells are multipotent stem cells. Mesenchyme is embryonic connective tissue that is derived from the mesoderm and that differentiates into hematopoietic and connective tissue. MSCs can be obtained from both marrow and non-marrow tissues, such as adult muscle side-population cells or the Wharton's jelly present in the umbilical cord.

Substantially homogenous populations of cells (e.g., 70, 80, 90, 95, 98, 99 or 100% homogenous) can be used in the methods and compositions of the disclosure.

A number of factors are known to cause differentiation of stem cells or progenitor cells along a directed lineage specific for various tissues. Non-limiting examples of bioactive molecules include activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation sphingolipid G-protein coupled receptor-1 (EDG1), ephrins, Epo, HGF, TNF-α, TGF-β, PD-ECGF, PDGF, IGF, IL8, growth hormone, fibrin fragment E, FGF-5, fibronectin, fibronectin receptor α₅β₁, Factor X, HB-EGF, HBNF, HGF, HUAF, heart derived inhibitor of vascular cell proliferation, IFN-gamma, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPARγ, PPARγ ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol (monobutyrin), and nicotinic amide.

Soluble factors that have been thought to play a role in morphogenetic capacity include hepatocytes growth factor (HGF) and epidermal growth factor (EGF) receptor ligands, which have been shown to induce branching tubular structures in epithelial cells cultured in collagen gels.

The term “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew its line or to produce progeny cells which will differentiate into fibroblasts or a lineage-committed progenitor cell and its progeny, which is capable of self-renewal and is capable of differentiating into a parenchymal cell type. Unlike pluripotent stem cells, lineage-committed progenitor cells are generally considered to be incapable of giving rise to numerous cell types that phenotypically differ from each other. Instead, they give rise to one or possibly two lineage-committed cell types.

The term “de-differentiation” is familiar to the person skilled in the relevant art. In general de-differentiation signifies the regression of lineage committed cell to the status of a stem cell, for example, by “inducing” a de-differentiated phenotype. For example, as described further herein KLF4, OCT4, SOX2, c-MYC or n-MYC, and Nanog can induce de-differentiation and induction of mitosis in lineage committed mitotically inhibited cells.

Stem cells are cells capable of differentiation into other cell types, including those having a particular, specialized function (e.g., tissue specific cells, parenchymal cells and progenitors thereof). Progenitor cells (i.e., “multipotent”) are cells that can give rise to different terminally differentiated cell types, and cells that are capable of giving rise to various progenitor cells. Cells that give rise to some or many, but not all, of the cell types of an organism are often termed “pluripotent” stem cells, which are able to differentiate into any cell type in the body of a mature organism, although without reprogramming they are unable to de-differentiate into the cells from which they were derived. As will be appreciated, “multipotent” stem/progenitor cells (e.g., neural stem cells) have a narrower differentiation potential than do pluripotent stem cells. Another class of cells even more primitive (i.e., uncommitted to a particular differentiation fate) than pluripotent stem cells are the so-called “totipotent” stem cells (e.g., fertilized oocytes, cells of embryos at the two and four cell stages of development), which have the ability to differentiate into any type of cell of the particular species. For example, a single totipotent stem cell could give rise to a complete animal, as well as to any of the myriad of cell types found in the particular species (e.g., humans).

The generation of patient-specific pluripotent stem cells has the potential to dramatically speed the implementation of stem cells into clinical use to treat degenerative diseases.

Technologies including Somatic Cell Nuclear Transfer (SCNT) and Cell Fusion would allow for such cells, but are fraught with issues that might prevent them from being put into clinical use. The disclosure provides methods to employ easily donated dermal fibroblasts from a single patient (e.g., autologous) and generate Human Induced Pluripotent Stem (hiPS or iPS) Cells by expression of a set of de-differentiation factors comprising (i) KLF4, OCT4, SOX2, c-MYC or n-MYC, NANOG or any combination thereof; (ii) KLF4, OCT4, SOX2, c-MYC or n-MYC, and NANOG; and (iii) KLF4, OCT4, SOX2, and NANOG. The cell lines generated are physiologically and morphologically indistinguishable from Human Embryonic Stem Cells (HESC) generated from the inner cell mass of a human embryo. hiPS cells share a nearly identical gene expression profile with two established HESC lines.

Human fibroblast (or other somatic cells) can be isolated from a subject and de-differentiated to induced pluripotent stem cells (iPSCs). The disclosure uses a plurality of de-differentiation factors for de-differentiating lineage committed cells to a more pluripotent or omnipotent cell type. As used herein a de-differentiation factor comprises a polynucleotide, polypeptide or small molecule. Exemplary de-differentiation factors comprising a polynucleotide are selected from the group consisting of a polynucleotide encoding a NANOG polypeptide, a c-MYC or n-MYC polypeptide, a KLF4 polypeptide, a SOX2 polypeptide or OCT4 polypeptide. Exemplary polypeptides comprise NANOG, c-MYC or n-MYC, KLF4, SOX2 or OCT4 polypeptides or polypeptides that increase the expression of any of the foregoing. Useful small molecule de-differentiation factors include molecules that stimulate the transcription or activity of an endogenous Nanog, c-Myc or n-Myc, Klf4, Sox4 or Oct4 polynucleotide or polypeptide, respectively.

A method to de-differentiate cells by expression of KLF4, OCT4, SOX2, c-MYC or n-MYC, NANOG or any combination thereof is presented. The nucleic acid and amino acid sequences of mouse and human KLF4, OCT4, SOX2, c-MYC or n-MYC, NANOG or any combination thereof are known in the art. The disclosure demonstrates that transfection with KLF4, OCT4, SOX2, c-MYC or n-MYC, and NANOG results in a de-differentiation of committed fibroblasts (e.g., dermal fibroblasts) to a pool of proliferating stem cells that are capable of re-differentiating into several cell types (including lineage committed neuronal cells).

In addition to the expression of a nucleic acid encoding an KLF4, OCT4, SOX2, c-MYC or n-MYC, and/or NANOG polypeptide, the disclosure contemplates that any agent which increase the expression and/or activity of an endogenous KLF4, OCT4, SOX2, c-MYC or n-MYC, NANOG or any combination thereof can be used in the methods of the disclosure to promote de-differentiation.

Nanog is a gene expressed in embryonic stem cells (ESCs) and plays a role in maintaining pluripotency. NANOG is thought to function with SOX2. Human NANOG protein (see, e.g., Accession number NP_079141, incorporated herein by reference) is a 305 amino acid protein with a homeodomain motif that is localized to the nuclear component of cells. Similar to murine NANOG, N-terminal region of human NANOG is rich in Ser, Thr and Pro residues and the C-terminus comprises Trp repeats. The homeodomain in human NANOG ranges from about residue 95 to about residue 155. Homologs of human NANOG are known.

Oct-4 (Octamer-4) is a homeodomain transcription factor of the POU family and regulates the expression of numerous genes (see, e.g., J. Biol. Chem., Vol. 282, Issue 29, 21551-21560, Jul. 20, 2007, incorporated herein by reference). Homologs of human Oct-4 are known as set forth in the following accession numbers NP_038661.1 and NM 013633.1 (Mus musculus), NP_001009178 and NM 001009178 (Rattus norvegicus), and NP_571187 and NM 131112 (Danio rerio), which are incorporated herein by reference.

SRY (sex determining region Y)-box 2, also known as SOX2, is a transcription factor that plays a role in self-renewal of undifferentiated embryonic stem cells and transactivation of Fgf4 as well as modulating DNA bending (see, e.g., Scaffidi et al. J. Biol. Chem., Vol. 276, Issue 50, 47296-47302, Dec. 14, 2001, incorporated herein by reference). Homologs of human SOX2 are known.

Kruppel-like factor 4, also known as KLF4 plays a role in stem cell maintenance and growth. Homologs of human KLF4 are known and include NP_034767, NM_010637 (Mus musculus), which are incorporated herein by reference.

The MYC family of cellular genes is comprised of c-myc, N-myc, and L-myc, three genes that function in regulation of cellular proliferation, differentiation, and apoptosis (Henriksson and Luscher 1996; Facchini and Penn 1998). Although myc family genes have common structural and biological activity. N-Myc is a member of the MYC family and encodes a protein with a basic helix-loop-helix (bHLH) domain. The genomic structures of C-myc and N-myc are similarly organized and are comprised of three exons. Most of the first exon and the 3′ portion of the third exon contain untranslated regions that carry transcriptional or post-transcriptional regulatory sequences. N-myc protein is found in the nucleus and dimerizes with another bHLH protein in order to bind DNA. Homologs and variants of the Myc family of proteins are known in the art.

cDNA coding for the human oct4 (pour5f1), sox2, klf4, c-myc (or n-myc) and nanog, variants and homologs thereof can be cloned and expressed using techniques known in the art. Using the sequences set forth in the accession numbers above and available to one of skill in the art, one or more de-differentiation factors can be cloned into a suitable vector for expression in a cell type of interest.

Cells can be engineered using any of a variety of vectors including, but not limited to, integrating viral vectors, e.g., retrovirus vector or adeno-associated viral vectors; or non-integrating replicating vectors, e.g., papilloma virus vectors, SV40 vectors, adenoviral vectors; or replication-defective viral vectors. Where transient expression is desired, non-integrating vectors and replication defective vectors may be used, since either inducible or constitutive promoters can be used in these systems to control expression of the gene of interest. Where the vector is a non-integrating vector, such vectors can be lost from cells by dilution after reprogramming, as desired. An example of a non-integrating vector includes Epstein-Barr virus (EBV) vector. Alternatively, integrating vectors can be used to obtain transient expression, provided the gene of interest is controlled by an inducible promoter. Other methods of introducing DNA into cells include the use of liposomes, lipofection, electroporation, a particle gun, or by direct DNA injection.

Conventional recombinant DNA techniques are used in the methods of the disclosure. For example, conventional recombinant DNA techniques are used to introduce the desired polynucleotide (e.g., KLF4, OCT4, SOX2, c-MYC or n-MYC, NANOG or any combination thereof) into differentiated cells to de-differentiate the cells into stem cells. The precise method used to introduce a polynucleotide is not critical to the disclosure. For example, physical methods for the introduction of polynucleotides into cells include microinjection and electroporation. Chemical methods such as co-precipitation with calcium phosphate and incorporation of polynucleotides into liposomes are also standard methods of introducing polynucleotides into mammalian cells. For example, DNA or RNA can be introduced using standard vectors, such as those derived from murine and avian retroviruses (see, e.g., Gluzman et al., 1988, Viral Vectors, Cold Spring Harbor Laboratory, Cold Spring Harbor, N.Y.). Standard recombinant molecular biology methods are well known in the art (see, e.g., Ausubel et al., 1989, Current Protocols in Molecular Biology, John Wiley & Sons, New York), and viral vectors for gene therapy have been developed and successfully used clinically (Rosenberg, et al., 1990, N. Engl. J. Med, 323:370). Other methods, such as naked polynucleotide uptake from a matrix coated with DNA are also encompassed by the disclosure (see, for example, U.S. Pat. No. 5,962,427, which is incorporated herein by reference).

Somatic cells, such as fibroblasts, are transformed or transfected with a polynucleotide encoding a de-differentiation factor(s), e.g., DNA, controlled by or in operative association with one or more appropriate expression control elements such as promoter or enhancer sequences, transcription terminators, polyadenylation sites, among others, and may further include a detectable marker. Following the introduction of the foreign DNA, engineered cells may be allowed to grow in enriched media and then switched to selective media.

The disclosure provides a method of producing vascularized tissue in vitro comprising transforming a pluripotent stem cell with a vector encoding one or more transcription factors that induce differentiation of the stem cell down a particular lineage path under the control of an inducible promoter to obtain parenchymal-inducible stem cells; combining the parenchymal-inducible stem cells with pluripotent stem cells lacking the vector under suitable co-culture conditions; and inducing the pluripotent stem cells down a vascular committed lineage pathway and either before, during or after inducing vacularization inducing expression of the one or more transcription factors in the parenchymal-inducible stem cells to cause the parenchymal-inducible stem cells to become parenchymal cells, wherein a vascularized tissue is obtain.

In some embodiments, the cells are cultures in or on a biocompatible material. A biocompatible matrix or gel may be of any material and/or shape that allows cells to attach to it (or can be modified to allow cells to attach to it) and allows cells to grow in more than one layer. A number of different materials may be used to form the matrix, including but not limited to: nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), nitrocellulose, cotton, polyglycolic acid (PGA), collagen (in the form of sponges, braids, or woven threads, and the like), cat gut sutures, cellulose, gelatin, or other naturally occurring biodegradable materials or synthetic materials, including, for example, a variety of polyhydroxyalkanoates. Any of these materials may be woven into a mesh, for example, to form the three-dimensional framework or scaffold. The pores or spaces in the matrix can be adjusted by one of skill in the art to allow or prevent migration of cells into or through the matrix material.

The three-dimensional framework, matrix, hydrogel, and the like, can be molded into a form suitable for the tissue to be replaced or repaired. For example, various techniques are known wherein a biocompatible matrix can be molded to form tubes, channels, islands, wells, and various shapes.

The term “polynucleotide sequence” can be the alphabetical representation of a polynucleotide molecule. This alphabetical representation can be input into databases in a computer having a central processing unit and used for bioinformatics applications such as functional genomics and homology searching.

Similarly, the term “polypeptide sequence”, “peptide sequence” or “protein sequence” can be the alphabetical representation of a polypeptide molecule. This alphabetical representation can be input into databases in a computer having a central processing unit and used for bioinformatics applications such as functional proteomics and homology searching.

As used herein, the term “recombinant expression system” refers to a genetic construct or constructs for the expression of certain genetic material formed by recombination.

As used herein, the term “recombinant protein” can refer to a polypeptide or peptide which is produced by recombinant DNA techniques, wherein generally, DNA encoding the polypeptide or peptide is inserted into a suitable expression vector which is in turn used to transform a host cell to produce the heterologous polypeptide or peptide.

As used herein, the term “subject” is intended to mean any animal. In some embodiments, the subject may be a mammal; in further embodiments, the subject may be a bovine, equine, feline, murine, porcine, canine, human, or rat.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, Calif.), LIPOFECTAMINE® (Invitrogen), FUGENE® (Roche Applied Science, Basel, Switzerland), JETPEI™ (Polyplus-transfection Inc., New York, N.Y.), EFFECTENE® (Qiagen, Valencia, Calif.), DREAMFECT™ (OZ Biosciences, France) and the like), or electroporation. Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals. Standard recombinant DNA and molecular cloning techniques used herein are well known in the art and are described in Sambrook, J., Fritsch, E. F. and Maniatis, T., Molecular Cloning: A Laboratory Manual, 2nd ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N.Y., (1984); and by Ausubel, F. M. et. al., Current Protocols in Molecular Biology, Greene Publishing and Wiley-Interscience (1987) each of which are hereby incorporated by reference in its entirety. Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

The terms “treat”, “treating” and “treatment”, as used herein, refers to ameliorating symptoms associated with a disease or disorder, including preventing or delaying the onset of the disease or disorder symptoms, and/or lessening the severity or frequency of symptoms of the disease or disorder.

Where in vitro engineered vascularized tissue is generated according to the methods and compositions of the disclosure transplantation of the tissue can be performed as follows. Surgery is performed on the recipient subject to expose the site to be treated, repaired and/or replaced. The in vitro engineered tissue is implanted directly into or adjacent to the recipient subject's tissue to be treated, repaired or replaced to result in the formation of chimeric tissue.

The implanted in vitro engineered tissue is allowed to grow within the recipient under conditions that allow the tissue to form vascular connections with the host. Suitable conditions may include the use of pre or post-operative procedures to prevent rejection of the implant as well as the administration of factors (e.g., pleotrophin, FGF1, GNDF, and the like) that stimulate tubulogenesis and/or morphogenesis of the in vitro engineered tissue. Immunosuppression techniques (in the absence or combined with genetically engineered techniques) such as cyclosporin A (CSA) to prevent rejection of the donor tissue are known in the art.

As used herein, the term “vector” can refer to a nucleic acid construct deigned for transfer between different hosts, including but not limited to a plasmid, a virus, a cosmid, a phage, a BAC, a YAC, etc. In some embodiments, a “viral vector” is defined as a recombinantly produced virus or viral particle that comprises a polynucleotide to be delivered into a host cell, either in vivo, ex vivo or in vitro. In some embodiments, plasmid vectors can be prepared from commercially available vectors. In other embodiments, viral vectors can be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct can refer to the polynucleotide comprising the retroviral genome or part thereof, and a gene of interest. Further details as to modern methods of vectors for use in gene transfer can be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.). In one aspect, the promoter is a pol III promoter.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and ‘Vector” can be used interchangeably. However, the disclosure is intended to include such other forms of expression vectors, such as viral vectors (e.g., replication defective retroviruses, adenoviruses and adeno-associated viruses), which serve equivalent functions. Typically, the vector or plasmid contains sequences directing transcription and translation of a relevant gene or genes, a selectable marker, and sequences allowing autonomous replication or chromosomal integration. Suitable vectors comprise a region 5’ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Typically, the vector or plasmid contains sequences directing transcription and translation of a gene fragment, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention including, but not limited to, lac, ara, tet, trp, IPL, IPR, T7, tac, and trc (useful for expression in Escherichia coli and Pseudomonas); the amy, apr, npr promoters and various phage promoters useful for expression in Bacillus subtilis, and Bacillus licheniformis; nisA (useful for expression in gram positive bacteria, Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)). Termination control regions may also be derived from various genes native to the preferred hosts.

The following examples are intended to illustrate but not limit the disclosure. While they are typical of those that might be used, other procedures known to those skilled in the art may alternatively be used.

### Examples

iMB-VO and iN-VO generation. To generate iMB-VOs or iN-VOs, 9,000 iMB or iN cells, respectively, were seeded in ULA 96-well plates for embryoid body (EB) formation. EBs were grown in EB media (DMEM/F12+20% KOSR+50 mM Y-27632) for 2 days and then transferred into an ULA 6-well plate, with about 8-12 EBs/well. This media was then replaced with 2 mL of Mesoderm Induction Media (N2/B27+BMP4+CHIR), with the addition of doxycycline to introduce either the muscle or neural compartment for iMB-VOs or iN-VOs, respectively. Doxycycline was spiked into the media for every media change onwards.

Organoids were then cultured at 37° C., 5% CO₂ for 3 days. Three days later, the medium was replaced with 2 mL of Vascular Induction Media (N2/B27+VEGF+FSK) per well and cultured at 37° C., 5% CO₂. This media was replaced 24 hours later and cultured at 37° C., 5% CO₂ for 24h. On day 7 of organoid formation, organoids were encapsulated in a blend of Matrigel and collagen and then cultured in Vascular Maturation Media (StemPro+15% FBS+VEGF+bFGF). Organoids were cultured at 37° C., 5% CO₂ and Vascular Maturation Media was replaced every 3 days. For long-term growth of iN-VOs, Vascular Maturation Media was supplemented with BDNF and NT-3 from day 15 and onwards.

Plasmid Construction. The piggyBac transposon plasmids for inducible overexpression of TFs were constructed using the backbone from 138-dCas9-Dnmt3a (Addgene Plasmid #84570) (Liu et al. 2016). The backbone plasmid was digested with NdeI and NsiI to remove the neomycin resistance cassette and was replaced with a puromycin resistance cassette using multi-element Gibson assembly. This puromycin resistant plasmid was then digested with NheI and AgeI to remove the dCas9-DNMT3A fusion sequence and replaced with the TF and fluorescent protein sequences separated by 2A peptide sequences using a multi-element Gibson assembly. TF and fluorescent protein sequences were amplified from plasmids:

### Fla_NEUROD1_P2A_Hygro_Barcode (Addgene Plasmid #120466),

EF1a_ASCL1_P2A_Hygro_Barcode (Addgene Plasmid #120427), phDLX2 N174 (Addgene Plasmid #60860) (Victor et al. 2014),
EF1a_MYOD1_P2A_Hygro_Barcode (Addgene Plasmid #120464), pBS-hBAF60C (Addgene Plasmid #21036) (Wang et al. 1996),
Fla_mCherry_P2A_Hygro_Barcode (Addgene Plasmid #120426), and pEGIP (Addgene Plasmid #26777).

To construct a plasmid expressing the hyperactive piggyBac transposase (Yusa et al. 2011), the sequence for the enzyme was obtained as a synthesized double-stranded DNA fragment (Integrated DNA Technologies). This was cloned into an in-house plasmid using Gibson assembly, such that the expression of the transposase is driven by a CAG promoter.

The Gibson assembly reactions were set up as follows: 100 ng digested backbone, 3:10 molar ratio of insert, 2×Gibson assembly master mix (New England Biolabs), H2O up to 20 μl. After incubation at 50° C. for 1 h, the product was transformed into One Shot Stbl3 chemically competent Escherichia coli (Invitrogen). A fraction (150 μL) of cultures was spread on carbenicillin (50 μg/ml) LB plates and incubated overnight at 37° C. Individual colonies were picked, introduced into 5 ml of carbenicillin (50 μg/ml) LB medium and incubated overnight in a shaker at 37° C. The plasmid DNA was then extracted with a QIAprep Spin Miniprep Kit (Qiagen), and Sanger sequenced to verify correct assembly of the vector. Following verification of the vector, larger amounts of plasmid were obtained by seeding 150 μl of bacterial stock into 150 ml of LB medium containing carbenicillin (50 μg/ml) and incubating overnight in a shaker at 37° C. for 16-18 h. The plasmid DNA was then extracted using a Plasmid Maxi Kit (Qiagen).

Cell Culture. H1 hESCs were maintained under feeder-free conditions in mTeSR1 medium (Stem Cell Technologies). Prior to passaging, tissue-culture plates were coated with growth factor-reduced Matrigel (Corning) diluted in DMEM/F-12 medium (Thermo Fisher Scientific) and incubated for 30 minutes at 37° C., 5% CO₂. Cells were dissociated and passaged using the dissociation reagent Versene (Thermo Fisher Scientific).

Generation of Clonal Inducible Overexpression Lines. hESC cells at 50-75% confluency from 3 wells of a 6-well plate were passaged using Versene. The cells were spun at 300 rcf for 5 minutes to obtain a cell pellet and this pellet was resuspended in a buffer containing 100 μl of P3 Nucleofector Solution (Lonza) and up to a maximum of 15 μl of a 1:1 mix of transposon vector plasmid to transposase plasmid by mass. This solution was loaded into a single Nucleovette (Lonza) and electroporated using the CB-150 pulse program on the 4D Nucleofector system (Lonza). After nucleofector run completion, 500 μl of pre-warmed mTeSR1 containing 10 μM Y27632 (Tocris Bioscience) was added to the Nucleovette and incubated at room temperature for 5 minutes. The cells were then removed from the Nucleovette using a Pasteur pipette and transferred dropwise into a 10 cm plate coated with growth-factor reduced Matrigel as previously described and containing pre-warmed mTeSR1 with 10 μM Y27632.

Medium was then changed daily, and 48 hours after nucleofection cells were maintained under puromycin (Thermo Fisher Scientific) selection at 0.75 μg/ml. After approximately 7-10 days of culture post-nucleofection, colonies were large enough for clonal selection. To pick clonal lines, cells were treated with Versene for 3 minutes, Versene was aspirated and the plate was filled with DMEM/F-12 with 1% antibiotic-antimycotic (Thermo Fisher Scientific). Individual colonies were then carefully scraped under a microscope and transferred into individual wells of a 24-well plate coated with growth-factor reduced Matrigel and containing pre-warmed mTeSR1. These individually picked clones were expanded, aliquots were frozen in mFreSR (Stemcell Technologies) and validated by differentiation to relevant cell types. One validated clone from each line was chosen for further experiments. All clones were maintained in mTeSR1 under selection with puromycin at 0.75 μg/ml.

2D Differentiation of Inducible Overexpression hESC lines. Clonal lines overexpressing NEUROD1 were differentiated following a previously described protocol (Zhang et al. 2013). Briefly, cells were passaged as single cells using Accutase (Innovative Cell Technologies) and plated in mTeSR1 at a density of 4-5×105 cells per well of a 6-well plate. The following day medium was changed to DMEM/F12 containing N2 supplement (Thermo Fisher Scientific), MEM non-essential amino acids (Thermo Fisher Scientific), 0.2 μg/ml mouse laminin (Invitrogen), 10 ng/ml BDNF (Peprotech), 10 ng/ml NT3 (Peprotech), 0.75 μg/ml puromycin and 1 μg/ml doxycycline (Sigma Aldrich) and cells were maintained in this medium for 2 days. On day 3 of differentiation cells were re-plated on Matrigel coated wells along with mouse glial cells in Neurobasal medium (Thermo Fisher Scientific) containing Glutamax (Thermo Fisher Scientific), B27 supplement (Thermo Fisher Scientific), 10 ng/ml BDNF, 10 ng/ml NT3 and 1 μg/ml doxycycline. From day 5 onward, 2 μM Ara-c (Sigma Aldrich) was added to the medium to inhibit astrocyte proliferation. 50% of the medium was subsequently changed every 2-3 days. Cells were maintained in culture for up to 3 weeks. For functional characterization and electrical measurements, cells were plated on Matrigel coated 6-well multi-electrode arrays (Axion Biosystems) with mouse glial cells and maintained in culture for up to 3 weeks.

Clonal lines overexpressing ASCL1 and DLX2 were differentiated following a previously described protocol (Yang et al. 2017). Briefly, cells were passaged as single cells using Accutase (Innovative Cell Technologies) and plated in mTeSR1 at a density of 4-5×105 cells per well of a 6-well plate.

The following day medium was changed to DMEM/F12 containing N2 supplement, MEM nonessential amino acids, 0.75 μg/ml puromycin and 1 μg/ml doxycycline and cells were maintained in this medium for 7-8 days, with the medium being changed every 2-3 days. 2 μM Ara-C was added to the medium on day 5 of differentiation. On day 7-8, the cells were passaged with Accutase and re-plated on Matrigel coated plates at a density of 4×105 cells per well of a 6-well plate in Neurobasal medium containing Glutamax, B27 supplement, 2 μM Ara-c and 1 μg/ml doxycycline. 50% of the medium was subsequently changed every 2-3 days. From day 15 onwards, medium was supplemented with 20 ng/ml BDNF and doxycycline was removed. For functional characterization and electrical measurements, cells were plated on Matrigel coated 6-well multi-electrode arrays (Axion Biosystems) with mouse glial cells and maintained in culture for up to 5 weeks.

Clonal lines overexpressing MYOD and BAF60C were differentiated to skeletal muscle following a process similar to a previously described protocol (Albini et al. 2013). Briefly, cells were passaged as single cells using Accutase and plated at a density of 4-5×105 cells per well of a 6-well plate. The following day, medium was changed to DMEM/F12 containing 15% fetal bovine serum (FBS, Thermo Fisher Scientific) and 1% anti-anti (Thermo Fisher Scientific). Medium was exchanged every 2 days. On day 5 of differentiation, medium was changed to DMEM/F12 containing 2% horse serum (Hyclone) and 1% anti-anti. Differentiating cells were cultured for 3 weeks.

iMB-VO and iN-VO Generation. hESCs were grown in one well of a 6-well plate till they were 80% confluent. This was sufficient to seed one ultra-low attachment (ULA) 96-well plate of embryoid bodies. To passage the cells, mTeSR was aspirated and the cells washed with PBS. 1 mL of Accutase was then added to the well and incubated at 37° C. incubator for 4-6 minutes. Cells were detached by tapping the sides of the plate. 1 mL of mTesr was then added to the well, and the detached cells were titurated with a 200 μl pipette to break up cell clumps and to obtain a single cell suspension. Cells were then spun down at 300 rcf for 5 minutes. Once the cells were pelleted, the supernatant was removed, and cells resuspended in EB medium—(DMEM-F12, 20% KOSR)+50 μM Y-27632—at a concentration of 72,000 cells/ml. 125 μL of this cell suspension, was dispensed into each well of an ULA 96 well plate. hESCs were cultured overnight at 37° C., 5% CO2, allowing them to aggregate into embryoid bodies (EBs).

EBs were grown for 1-3 days till 200-400 μm in diameter. Once this size, EBs were transferred into an ULA 6-well plate using a cut 200 μL pipette tip, with a maximum of ˜24 EBs per well of the ULA 6 well plate. Excess EB medium was carefully removed and 2 mL of Mesoderm Induction Media: N2/B27 medium—(1:1 DMEM/F12-Neurobasal, 100×N2, 50×B27)—+3 μM CHIR (Tocris), 30 ng/mL BMP4 (Peprotech), 1 μg/ml of doxycycline (dox); was added to each well. These were then cultured at 37° C., 5% CO₂ for 3 days.

Three days later, the medium was replaced with 2 mL of Vascular Induction Media: N2/B27 medium+100 ng/ml VEGF (Peprotech)+10 μM Forskolin (Sigma-Aldrich)+1 μg/ml dox per well; and cultured at 37° C., 5% CO₂. 24 hours later, medium was removed and replaced with 2 mL of N2/B27 media+100 ng/ml VEGF+10 μM Forskolin+1 μg/ml dox per well. Organoids were then cultured at 37° C., 5% CO₂ for 24 hours.

Organoids were then encapsulated in a blend of Matrigel and collagen (Mat-Col gel: 2 mg/mL Collagen (Advanced Biomatrix)+20% Matrigel). Briefly, parafilm wells were prepared by placing a piece of parafilm onto an empty 200 μL pipette tip box, and pressing into the tip cavities to create dimples. A 7×7 grid of wells was found to be optimal since it prevented organoids from drying and allowed sufficient organoids to be encapsulated for culturing in one 10 cm dish. The dimpled parafilm was then placed in a 10 cm dish. The Mat-Col Gel Blend was prepared and placed on ice.

Using a cut 200 μL pipette tip, organoids were transferred from the ULA 6 well plate, and placed individually into the parafilm wells. A maximum of ˜30 μL of media was transferred with each organoid to avoid overfilling of the parafilm well. Once all organoids were placed in individual wells, excess media was removed, leaving only the organoid in the well. 30 μL of the Mat-Col gel was added to each parafilm well. Individual organoids were checked to ensure encapsulation in the gel solution, and the 10 cm dish was then incubated at 37° C., 5% CO2 for 45 minutes for the gel blend to completely gelate.

Once gelation was complete, the encapsulated organoids were washed off the parafilm using Vascular Maturation Media: StemPro 34 Media+15% FBS+100 ng/ml VEGF+100 ng/ml bFGF+1 μg/ml dox. Once gel droplets were completely washed off from the parafilm, a cut 1000 μL pipette tip was used to transfer the organoids back to the original ULA 6 well plate used in the previous steps of the experiment. Organoids were then cultured at 37° C., 5% CO2 and medium replaced every 3 days using Vascular Maturation Media until day 15.

For long term culture of iN-VOs, at day 15, medium was changed to Vascular Maturation Media+20 ng/ml BDNF+20 ng/ml NT3+1 μg/ml dox. Medium was replaced every 3-5 days.

Animals. Housing, husbandry and all procedures involving animals used in this study were performed in compliance with protocols (#S16003) approved by the University of California San Diego Institutional Animal Care and Use Committee (UCSD IACUC). Mice were group housed (up to 4 animals per cage) on a 12:12 hr light-dark cycle, with free access to food and water in individually ventilated specific pathogen free (SPF) cages. All mice used were healthy and were not involved in any previous procedures nor drug treatment unless indicated otherwise. All studies performed in NOD.Cg-Prkdcscid Il2rgtm1Wjl/SzJ (NSG) mice and maintained in autoclaved cages.

In vivo Perfusion. iN-VOs were cultured for 30 days before being implanted subcutaneously into Rag2^{-/-};γc^{-/-} immunodeficient mice. To prepare the mice for subcutaneous implantation, the right hind-flank region was shaved and wiped down with povidone-iodine. Then, a one-inch, subcutaneous incision was made, and Day 30 iN-VOs suspended in Matrigel were placed inside the incision region using a cut pipette tip. These organoids were then matured for an extra 30 days in-vivo.

To test for proper perfusion of the vasculature, mice were given an intravenous injection of lysine fixable Texas-Red Dextran (1.25 mg per mouse, Thermo Fisher Scientific) and sacrificed after 15 minutes of allowing the dextran to pass through circulation. Organoids were retrieved from the subcutaneous region, fixed and whole-mount stained, as described below.

MEA Measurements. For 2D differentiated neurons, cells were plated on Matrigel coated 6-well multi-electrode arrays (CytoView MEA 6, Axion Biosystems) with mouse glial cells and maintained in culture for up to 3 weeks.

iN-VOs were not encapsulated in Mat-Col gel when preparing them for MEA measurements. MEA electrodes (CytoView MEA 6, Axion Biosystems) were spot-coated with 2% Matrigel and placed in a cell-culture incubator to incubate at 37° C. overnight. Because gel encapsulation prevented proper adhesion between the organoid and MEA well, the following day, one day-25 unencapsulated iN-VO was carefully put in the center of the MEA well with ˜50 μL of media. The organoid was left untouched for 2 hours, and then flooded with 0.5 mL of media. PBS was filled in the side compartments of the MEA plate to prevent cell media evaporation. The MEA plates were then left undisturbed for 5 days to ensure robust attachment to the well. MEA measurements were taken on day 30, 5 days after seeded onto the plate. To collect measurements, MEA plates were placed in the reader with the reader plate heater set to 37° C. and under 95% O2/5% CO2 air flow. Plates were allowed to equilibrate under these conditions for a minimum of 5 minutes before collecting spontaneous recordings for 4 minutes.

Electrical signals were collected and analyzed using AxIS Software (Axion Biosystems) with Spontaneous Neural configuration. Signals were filtered with a band-pass filter of 200 Hz-3 kHz. Spikes were detected with AxIS software using an adaptive threshold crossing set to 5.5 times the standard deviation of the estimated noise for each electrode.

In vitro electrical stimulation. To create chambers for electrical stimulation, custom designed chips consisting of a porous inner well and a solid outer well were fabricated via extrusion printing of a silicone elastomer (Dow Corning Dowsil SE 1700) on glass slides, on a custom 3D printer consisting of a three-axis gantry (AGB 10000, Aerotech) and pneumatic dispensers (Nordson Ultimus I). Chips were cured for at least two hours at 80° C. to fully crosslink the elastomer. Graphite rods were then inserted into these chips such that they were located at either end of the inner well and gaps were sealed with PDMS (Dow Corning, Sylgard 184). After sealing, chips were again cured for at least two hours at 80° C.

To encapsulate iMB-VOs, a solution composed of Fibrin (3 mg/ml, Sigma Aldrich)+20% Matrigel was prepared similarly to a previously described protocol (Rao et al. 2018). Up to five individual organoids were transferred into the inner well, excess medium removed and the space filled with the hydrogel mixed with thrombin (1 U/ml, Sigma-Aldrich). The hydrogel was allowed to fully gelate and crosslink at 37° C. for one hour, after which the outer well was filled with VO culture medium containing 1 μg/ml dox. For stimulated samples, wires were attached to the graphite rods and routed to Arduino Uno microcontrollers equipped with Motor Shields. The microcontrollers were programmed to provide chronic electrical stimulation at 0.4 V/mm, 1 Hz with a 2 ms on time. Encapsulated organoids were then cultured in 37° C., 5% CO₂ for eight days. On the second day after encapsulation, electrical stimulation was started and maintained for one week.

Immunostaining. Organoids were removed from the culture dish and added to a 1.5 mL centrifuge tube. Up to 20 organoids could be combined into one tube and used in subsequent steps. Excess medium was removed and organoids were washed once with 1 mL PBS. PBS was removed and 500 μL of 4% PFA solution was added to the microcentrifuge tube. Organoids were fixed at room temperature for 1 hour, protected from light. After 1 hour, PFA solution was removed and exchanged with 500 μL PBS. At this point, organoids could be stored in PBS at 4° C. protected from light for up to 1 month.

To block the organoids, and prepare them for immunostaining, PBS was removed and 500 μL of blocking buffer (3% FBS, 1% BSA, 0.5% Triton-X, 0.5% Tween) was added. The tube was placed into a tube rack and then onto an orbital shaker, shaking at 150 rpm for 2 hours to fully block and permeabilize the organoids. Blocking buffer was then removed and 100 μL of primary antibody diluted in blocking buffer was added. All antibodies used were diluted 1:100 in blocking buffer. The tube then was placed back onto the tube rack and onto an orbital shaker (LSE Orbital Shaker, Corning) at 4° C. The orbital shaker was set to 12 rpm and organoids incubated at 4° C. overnight.

After overnight incubation, blocking buffer was removed, and organoids washed with PBS-T (PBS+0.05% Tween) three times for 20 minutes. Organoids were placed on an orbital shaker set to 150 rpm during each PBS-T wash.

After washing in PBS-T, 100 μL of secondary antibodies diluted in blocking buffer were added. Organoids were incubated with the secondary antibodies at room temperature for 2 hours, while keeping the samples protected from light. After secondary staining was complete, organoids were washed with PBS-T three times for 20 minutes. Organoids were placed on an orbital shaker set to 150 rpm during each PBS-T wash.

Once secondary staining was complete, a coverslip was prepared for whole-mounting of the organoids. This was done by applying epoxy (Loctite Epoxy) to the non-adhesive surface of an iSpacer and then attaching the iSpacer to a coverslip. Within 5 minutes the iSpacer was bound to the coverslip. Using a cut 1000 μL pipette tip, 2-4 organoids were transferred to each iSpacer well. Excess PBS was removed and 50 μL of Fluromount G was added to each well. iSpacer cover was then peeled off and a second coverslip attached the exposed sticky side. Whole-mount samples could be stored in 4° C. protected from light for up to 8 months. Confocal images were taken using a LSM 880 with Airyscan Confocal Microscope (Zeiss).

All of the Primary and Secondary Antibodies used in this protocol are diluted in Blocking Buffer at a 1:100 dilution factor. anti-VE-Cadherin (D87F2, Cell Signaling Technologies), anti-MAP2 (HM-2, Sigma-Aldrich), anti-MYH (MF-20, DSHB), anti-PDGFR (AF385, R&D Systems) and anti-SMA (MAB1420, R&D Systems) were used for primary antibody staining. anti-Rabbit Alexa 405 (Thermo Fisher Scientific, A-31556), anti-Rabbit DyLight 550 (Thermo Fisher Scientific, 84541), and anti-Mouse Alexa 647 (Thermo Fisher Scientific, PIA32728) were used for secondary antibody staining.

For endothelial function Dil-acetylated low-density lipoprotein (Dil-ac-LDL) uptake assay, neuro-vascular organoids were incubated with 10 μg ml-1 Dil-ac-LDL (Thermofisher, L3484) for 6 hours and then washed several times in medium before immunostaining and imaging.

RNA Extraction and qRT-PCR. RNA was extracted from cells using the Qiazol and RNeasy Mini Kit (Qiagen) as per the manufacturer's instructions. The quality and concentration of the RNA samples was measured using a spectrophotometer (Nanodrop 2000, Thermo Fisher Scientific). cDNA was prepared using the Protoscript II First Strand cDNA synthesis kit (New England Biolabs) in a 20 μl reaction and diluted up to 1:2 with nuclease-free water.

qRT-PCR reactions were setup as: 2 μl cDNA, 400 nM of each primer, 2×iTaq Universal SYBR Supermix (Bio-Rad), H₂O up to 20 μl. qRT-PCR was performed using a CFX Connect Real Time PCR Detection System (Bio-Rad) with the thermocycling parameters: 95° C. for 3 min; 95° C. for 3 s; 60° C. for 20 s, for 40 cycles. All experiments were performed in triplicate and results were normalized against a housekeeping gene, GAPDH. Relative mRNA expression levels, compared with GAPDH, were determined by the comparative cycle threshold (ΔΔCT) method. The primers used for qPCR reactions are set forth in the priority document (Provisional Application No. 63/285,911, which are incorporated herein by reference).

Statistics. All statistics on gene expression qPCR plots were assayed via an unpaired two-tailed t-test. P values were assessed as significant as follows: **P≤0.01, ***P≤0.001, and ****P≤0.0001; ns=not significant. Statistical analysis was carried out using GraphPad Prism 8.

Single cell RNA-seq Processing. To dissociate organoids for single cell RNA-seq, 5-6 organoids were incubated in a 1 mL 20 U/mL Papain solution (Worthington, LS003126) for 30 minutes, passed through a 40 μm filter, spun down at 300 rcf for 5 minutes, and resuspended in 0.04% BSA solution. Cells were then loaded into the Chromium Chip B (10× Genomics) and single cell libraries were made using Chromium Single Cell 3′ Reagent Kits v3 workflow (10×Genomics). Fastq files were aligned to a hg19 reference and expression matrices generated using the count command in cellranger v3.0.1 (10×Genomics).

Data Integration and Clustering. Data integration was performed on the expression matrices from all 3 organoids: iN-VOs induced for 15 days; iN-VOs induced for 45 days; and iN-VOs which were not induced. Integration was done using the Seurat v3 pipeline (Butler et al. 2018). Expression matrices were filtered to remove any cells expressing less than 200 genes or expressing greater than 10% mitochondrial genes. DoubletFinder (McGinnis, Murrow, and Gartner 2019) was used to detect predicted doublets, and these were removed for downstream analysis. The expression matrix was then normalized for total counts, log transformed and scaled by a factor of 10,000 for each sample, and the top 4000 most variable genes were identified. Seurat was then used to find anchor cells and integrated all data sets, obtaining a batch-corrected expression matrix for subsequent processing. This expression matrix was scaled, and nUMI as well as mitochondrial gene fraction was regressed out. Principal component analysis (PCA) was performed on this matrix and 22 PCs were identified as significant using an elbow plot. The 22 significant PCs were then used to generate a k-nearest neighbors (kNN) graph with k=10. The kNN graph was then used to generate a shared nearest neighbors (sNN) graph followed by modularity optimization to find clusters with a resolution parameter of 0.8.

To classify cell types, the integrated dataset was mapped to annotated cell types in the Microwellseq Mouse Cell Atlas (Han et al. 2018) using Seurat label transfer on the intersection of genes in the mouse and organoid datasets, and further refined using cell type-specific marker genes. The results were visualized using UMAP dimensionality reduction on the first 22 PCs.

To assess tissue-specificity of endothelial cells, the endothelial cell cluster was subsetted and mapped using Seurat label transfer to tissue-specific endothelial cells from the Tabula Muris consortium. To confirm neuronal character of neurons from the neurovascular organoids, the neuron cluster was subsetted and mapped via Seurat label transfer to cells profiled during differentiation of neurons from pluripotent stem cells in 2D using NGN2 overexpression. For all mapping to a reference data set, the prediction score is assessed as the mean of the Seurat predicted identity score across all cells with a particular identity.

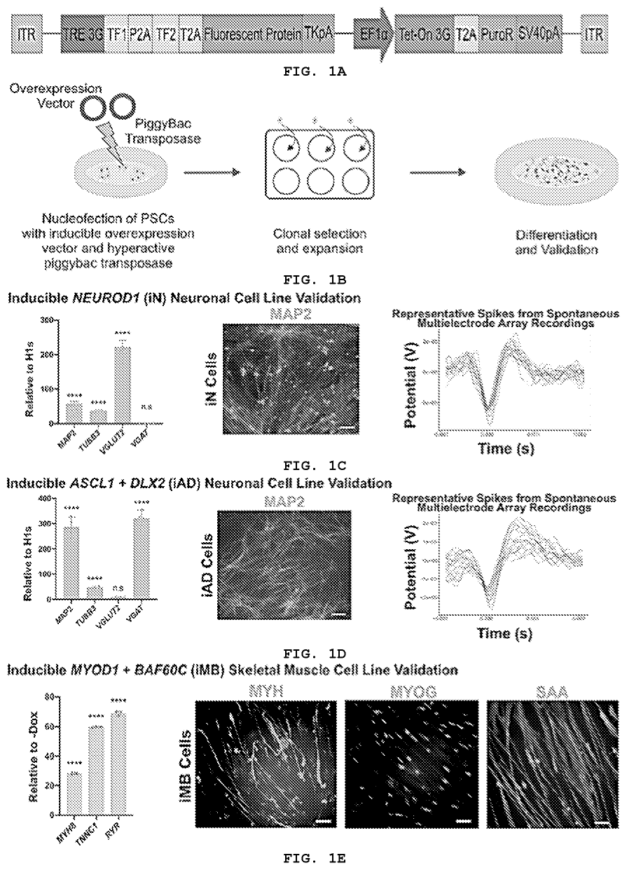

Inducible cell line construction and validation. For the inducible expression of transcription factors (TFs), a PiggyBac transposon-based overexpression vector was made that allowed a single vector to package the complete Tet-On system for doxycycline inducible expression, along with one or more TFs to be overexpressed in conjunction with a reporter fluorescent protein (FIG. 1A). To establish the utility of the PiggyBac overexpression platform, an array of cell lines was constructed and their ability to differentiate into functional tissue upon adding doxycycline validated. For neural differentiation, overexpression of NEUROD1 was chosen to generate glutamatergic excitatory neurons (Parekh et al., 2018; Zhang et al., 2013), and ASCL1+DLX2 for GABAergic inhibitory neuron differentiation (Yang et al., 2017). For mesodermal tissue differentiation, overexpression of MYOD1+BAF60C (Albini et al., 2013) for skeletal muscle differentiation was chosen. Stable, dox-inducible NEUROD1 (iN), ASCL1+DLX2 (iAD), and MYOD1+BAF60C (iMB) human embryonic stem cell (hESC) lines were generated by nucleofecting hESCs with the respective overexpression vector, along with a hyperactive PiggyBac transposase (Yusa et al., 2011).

For validation of neural differentiation of iN and iAD lines, qRT-PCR analysis demonstrated upregulation of neuronal markers MAP2 and TUBB3 for both iN and iAD cells (FIGS. 1C and 1D) compared with undifferentiated hESCs. iN neurons were confirmed to be glutamatergic via upregulation of excitatory marker VGLUT2 with no detectable expression of the inhibitory marker VGAT (FIG. 1C), while iAD neurons were confirmed to be GABAergic, via upregulation of VGAT with minimal expression of VGLUT2 (FIG. 1D). MAP2+ cells with classic neuronal morphology were confirmed by immunofluorescence for both iN and iAD cells (FIGS. 1C and 1D). Finally, iN and iAD neurons were confirmed to be functional by detection of spontaneous firing when co-cultured with glia and measured on a microelectrode array (MEA) at 3-5 weeks post induction (FIGS. 1C and 1D).

Skeletal muscle differentiation of iMB cells was validated by gene expression and immunofluorescence assays (FIG. 1E). qRT-PCR analysis confirmed upregulation of skeletal muscle markers MYH8, TNNC1, and RYR. Immunofluorescence confirmed the presence of characteristic spindle-shaped MYH+ and SAA+skeletal muscle morphology, along with MYOG+nuclei.

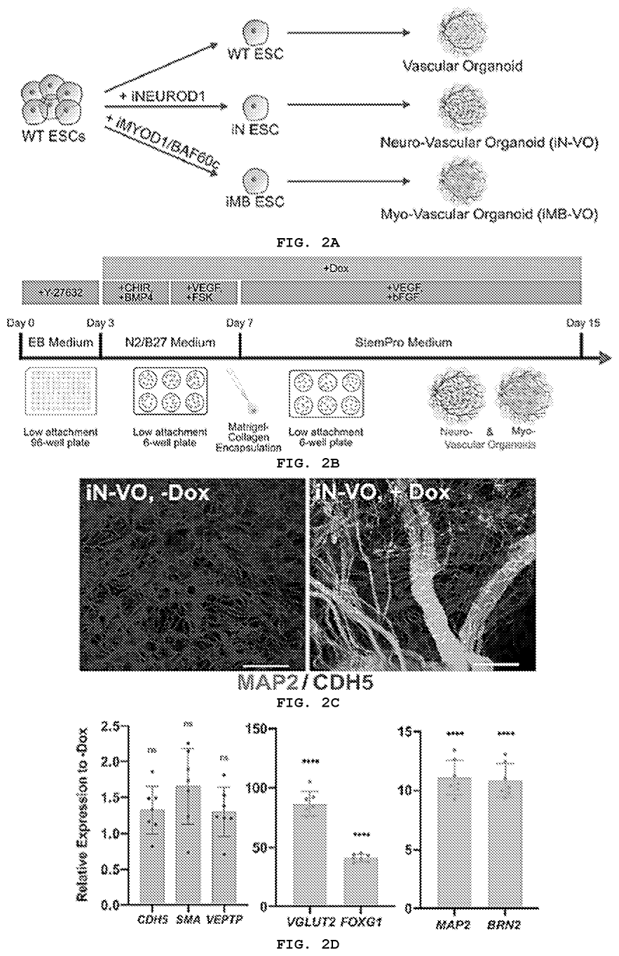

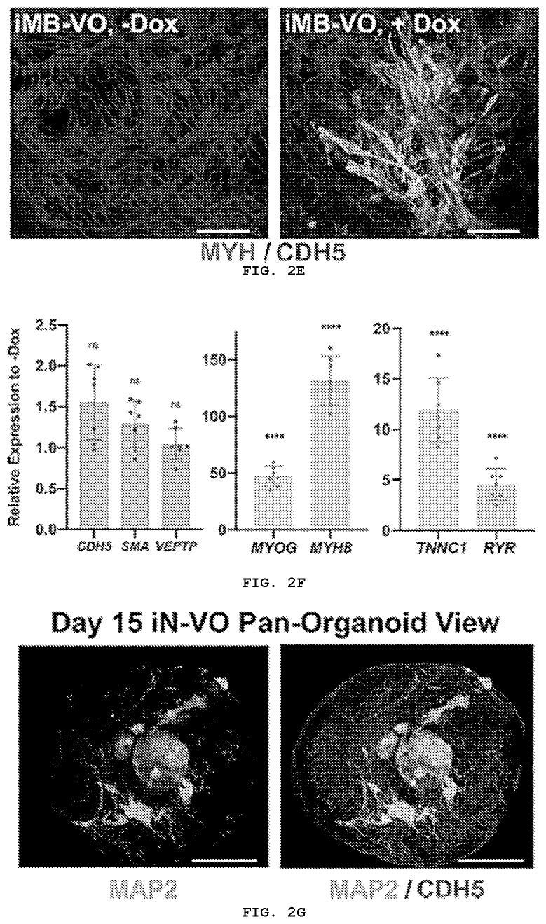

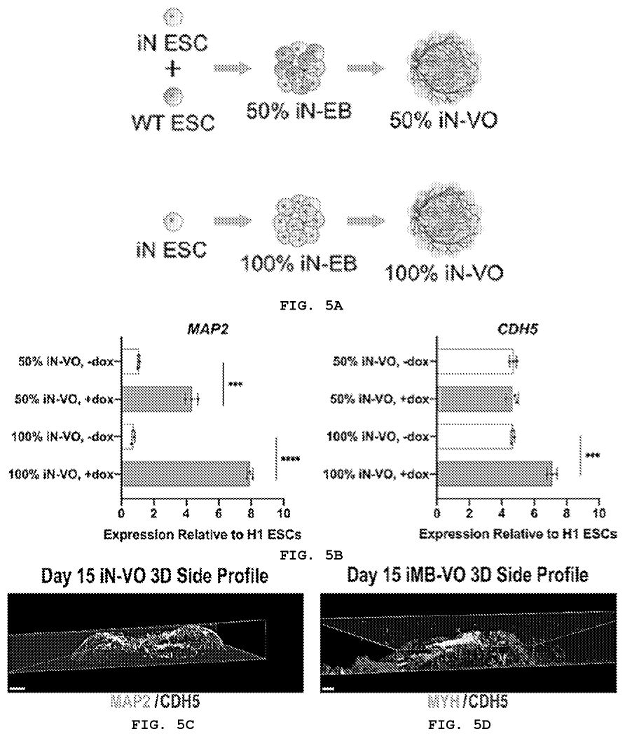

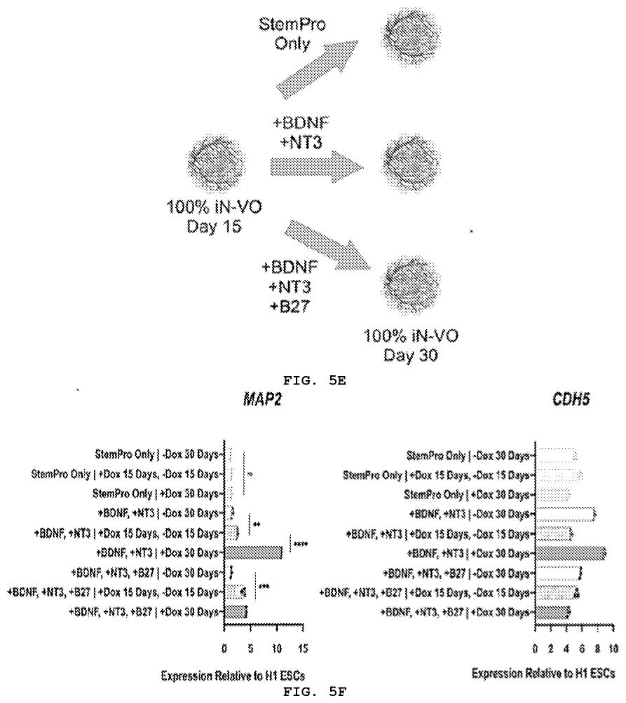

Constructing neuro-vascular and myo-vascular organoids. After clonal hESC lines were established, a modular, generalizable, TF overexpression-based strategy was developed to differentiate parenchymal cell types in to vascular organoids (VOs) (FIG. 2A). An optimization experiment was conducted to determine if wild-type ESCs are required for maintaining the vascular compartment in the organoids (FIG. 5A). Given the higher expression of MAP2 and comparable expression of CDH5, subsequent experiments were conducted with iN-VOs formed from 100% iN cells (FIG. 5B). Immunostaining was performed on both induced (+dox, iN-VOs) and uninduced (−dox, iN-VOs) organoids, confirming MAP2+ cells forming into bundle-like structures of neurons only in induced organoids, along with dense, interpenetrating CDH5+vascular networks in both conditions (FIG. 2C). Furthermore, pan-organoid confocal images show that endothelial networks span all layers of the organoid, as do neurons, although they are not uniformly distributed and form clusters and bundles of cells (FIGS. 2G and S15C). Further analysis via qRT-PCR showed an upregulation of the excitatory marker VGLUT2, along with cortical neuron markers BRN2 and FOXG1 (Zhang et al., 2013) in day 15 iN-VOs (FIG. 2D). Moreover, expression levels comparable with uninduced organoids were observed of the endothelial markers CDH5 and VEPTP, along with the smooth muscle marker SMA (FIG. 2D).

Once the formation of neuro-vascular organoids was established, experiments were performed to demonstrate if the system was compatible with other germ-layer or cross-lineage cell types. Using 100% iMB cells and the same protocol used to generate neuro-vascular organoids, iMB-VOs were grown (FIG. 2B). iMB-VOs had MYH+spindle-shaped cells (FIG. 2E), along with expression of skeletal muscle marker genes MYOG, MYH8, TNNC1, and RYR (FIG. 2F), confirming that the platform could also be used to grow vascularized tissue of mesodermal origin, specifically skeletal muscle. Pan-organoid tile-scan confocal images demonstrated MYH+skeletal muscle cells present in many layers of the myo-vascular organoid, albeit not uniformly distributed throughout the organoid (FIGS. 2H and 5D).

Comprehensive characterization of neuro-vascular organoids. Experiments were performed to further enable long-term culture of the neuro-vascular organoids beyond the initial 15 days (FIG. 5E). Media optimization experiments revealed that brain-derived neurotrophic factor (BDNF) and neurotrophin-3 (NT-3) supplementation from day 15 ensured long-term survival of neurons with reliable maintenance of vascular lineages (FIG. 5F).

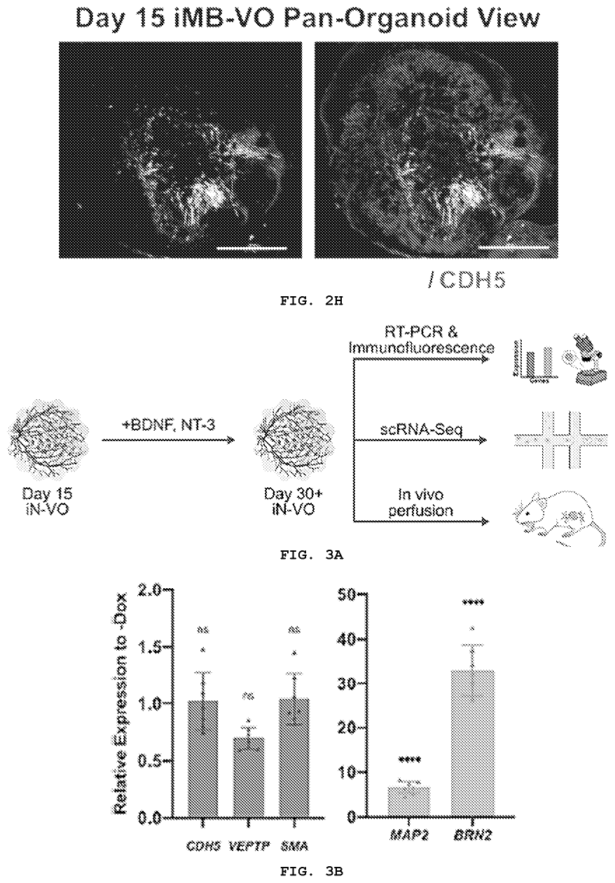

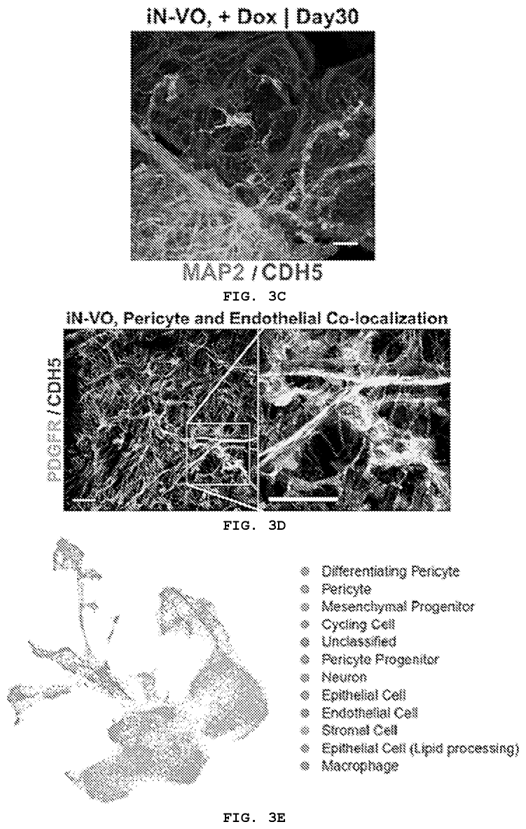

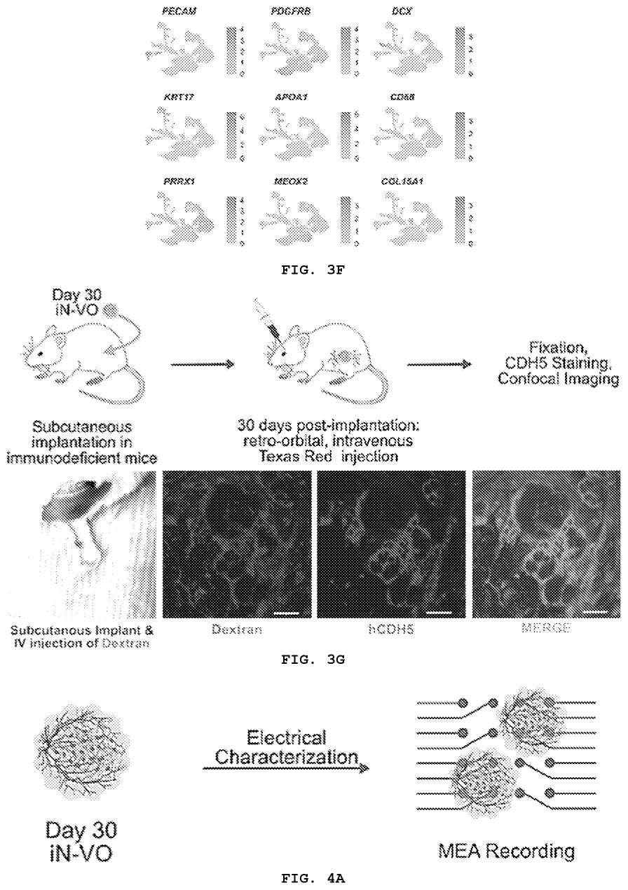

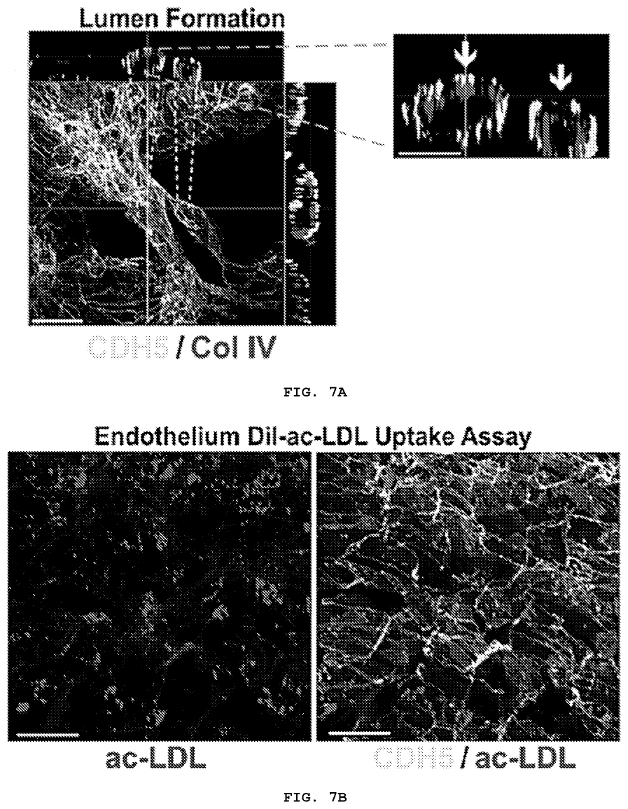

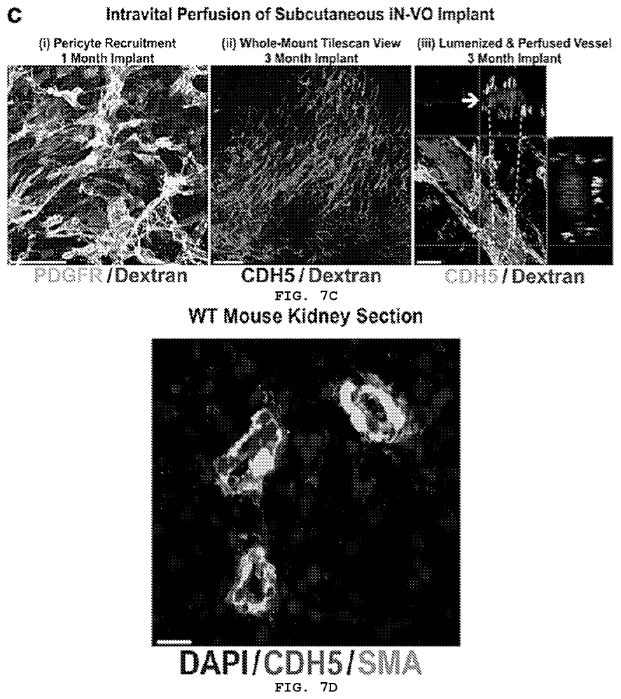

Upon determining an optimized protocol for long-term growth of iN-VOs, characterization of these long-term cultured neuro-vascular organoids was performed (FIG. 3A). qRT-PCR analysis showed an upregulation of the neuronal markers MAP2, VGLUT2, BRN2, and FOXG1 in day 30 iN-VOs (FIG. 3B), while maintaining the expression level of endothelial markers CDH5 and VEPTP, and smooth muscle marker SMA (FIG. 3B). Immunofluorescence imaging confirmed the presence of MAP2+neurons displaying robust neurite growth, along with interpenetrating CDH5+vascular networks through 30 days in culture (FIG. 3C). These organoids formed capillary networks that consisted of lumen-forming endothelial cells (FIG. 7A) that were tightly associated with pericytes (FIG. 3D).

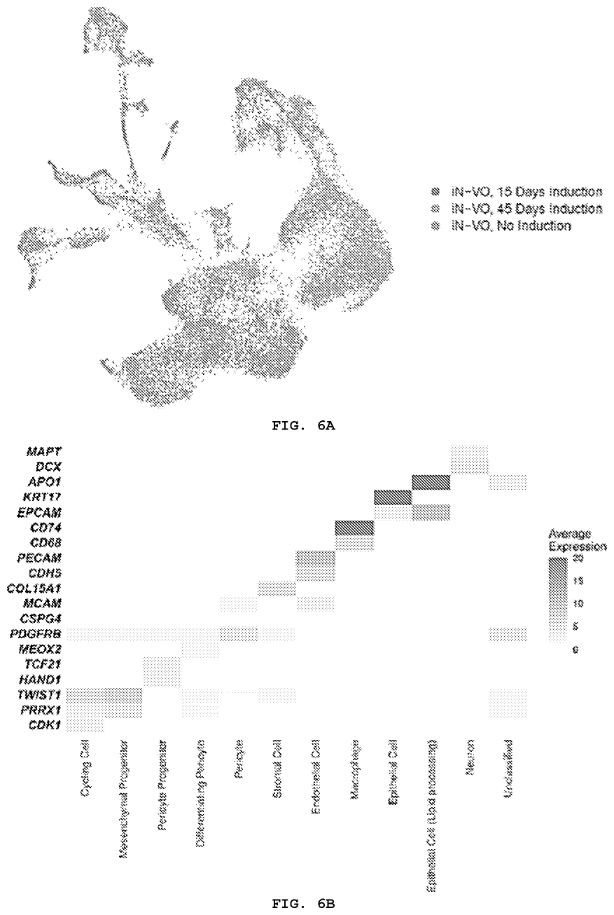

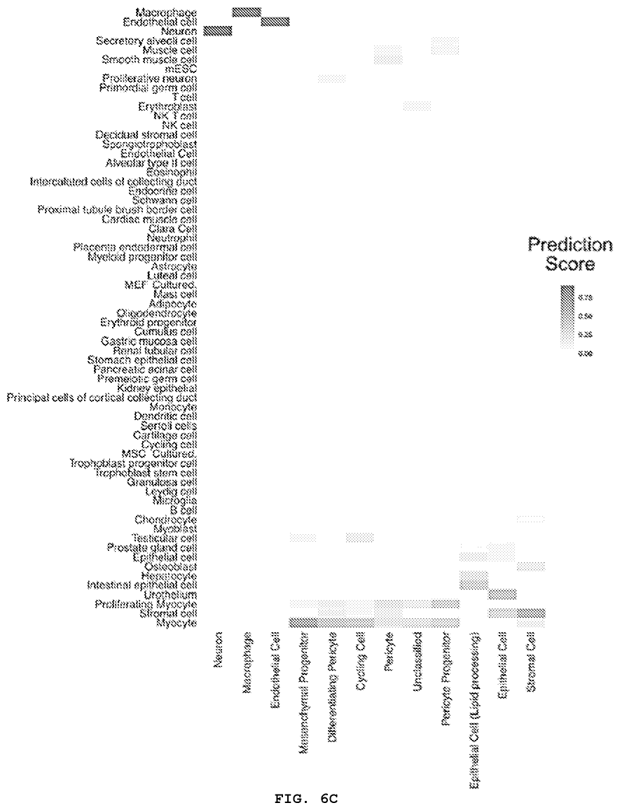

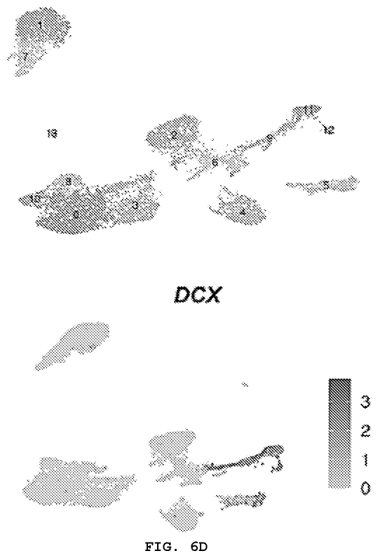

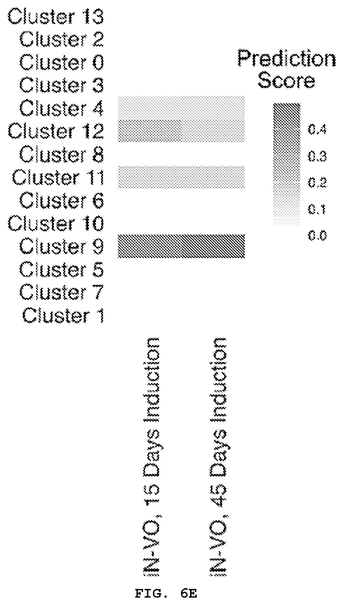

To characterize the cell-type composition of iN-VOs cultured for up to 45 days, single-cell RNA-sequencing (scRNA-seq) assays were performed. 26,959 cells were assayed across day 45 organoids grown under three conditions: 15 days of NEUROD1 overexpression (iN-VO, 15 days induction), 45 days of NEUROD1 overexpression (iN-VO, 45 days induction), and without overexpression (iN-VO, no induction). Using the Seurat pipeline (Butler et al., 2018), PRRX1+mesenchymal progenitors, MEOX2+differentiating pericytes, and cycling cells were identified and present in all three types of organoids and constituted a majority of each of the organoids (FIGS. 3E, 3F, 6A, and 6B). Functional vascular cell types PDGFRβ+ pericytes and PECAM1+endothelial cells were present in all types of organoids and constituted 16%-22% and 2.5%-6% of cells respectively (FIGS. 3E, 3F, 6A, and 6B). Endothelial and neural cells mapped with high fidelity to the endothelium and neurons from the Mouse Cell Atlas, respectively (FIG. 6C). To further validate their identity, neurons from the neuro-vascular organoids were mapped to DCX+neuronal clusters from a reference dataset profiling two-dimensional (2D) differentiation of neurons from hPSCs by NGN2 overexpression (Schornig et al., 2021) (FIGS. 6D and 6E). After molecular characteristics of the iN-VOs were assayed, the functionality of both neural and vascular cell types in the organoid were assessed. First endothelial function was assessed in vitro by confirming uptake of acetylated low-density lipoprotein by endothelial cells in the organoids (FIG. 7B). Further, the ability of the vascular networks to form perfusable blood vessels when implanted into mice was assessed. Specifically, iN-VOs were grown until day 30 in vitro, and then subcutaneously implanted in Rag2^{-/-};γc^{-/-} immunodeficient mice. Thirty and 90 days post implantation, intravenous (IV) injection of a Texas red dye followed by organoid extraction, fixation, and immunofluorescence staining showed co-localization of the dye with human CDH5+vascular cells and PDGFR+pericytes in the iN-VO (FIGS. 3G and 7C). Endothelium function in vivo was sustained for at least 90 days post implantation, as demonstrated by confocal imaging of lumenized and perfused CDH5+vessels (FIG. 7C(ii),(iii)). The absence of CDH5 signal in wild-type mouse kidneys stained with human-specific anti-CDH5 demonstrates the human specificity of the antibody (FIG. 7D), confirming that the perfused CDH5+vessels in the organoids are of human origin.

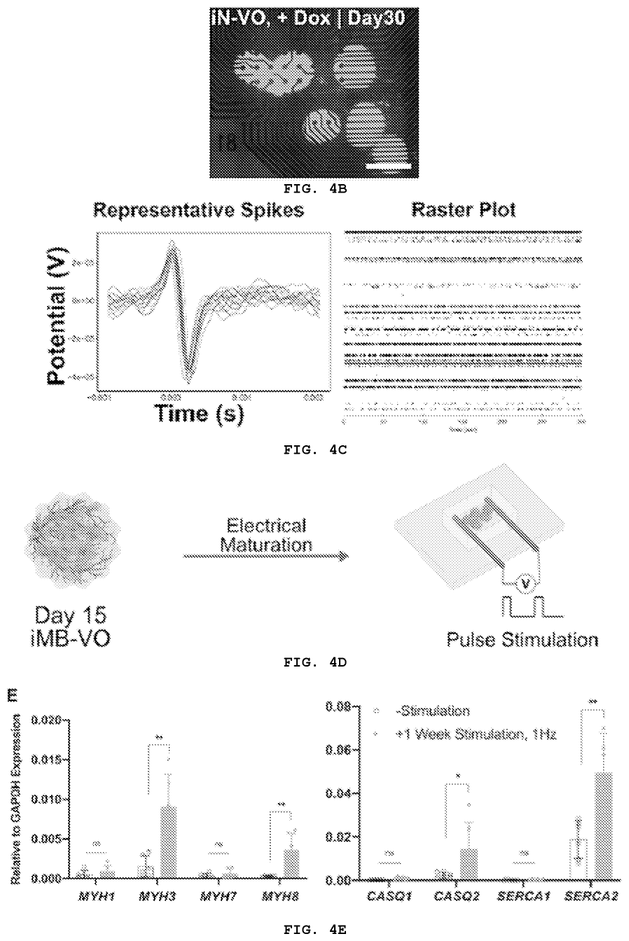

To assess functionality of neurons in the iN-VOs, the organoids were assayed for spontaneous electrical activity using MEAs (FIGS. 4A and 4B). Strikingly, while uninduced organoids displayed no spontaneous activity, spontaneous firing was repeatedly observed in iN-VOs (FIG. 4C), confirming the presence of functional neurons. This demonstrates the formation of functional neuro-vascular tissue with long-term culture capability.

In vitro maturation of myo-vascular organoids. The iMB-VO platform was able to differentiate vascularized skeletal muscle, maturation of this lineage in vitro is a long-standing challenge. To further mature the differentiated skeletal muscle, chronic electrical stimulation was applied (Khodabukus et al., 2019; Rao et al., 2018).

To subject organoids to stimulation, the organoids were encaspulated in a fibrin and Matrigel blend, and placed in a custom chip between two graphite rods. A pulsed constant-voltage stimulation was then applied for a week after encapsulation to drive maturation (FIG. 4D). Organoids were then assayed for gene expression of muscle and calcium-handling genes (FIG. 4E). In stimulated organoids, embryonic skeletal muscle myosin (MYH3) was upregulated, along with a small increase in expression of adult fast skeletal muscle myosin (MYH2). Additionally, the calcium-handling genes CASQ2 and SERCA2 were also highly upregulated. The iMB-VO approach is a promising method to generate mature, vascularized skeletal muscle tissue in vitro.

To assess functionality of neurons in the iN-VOs, the organoids was assessed for spontaneous electrical activity using MEAs (FIGS. 4A and 4B). Strikingly, while uninduced organoids displayed no spontaneous activity, spontaneous firing was repeatedly observed in iN-VOs (FIG. 4C), confirming the presence of functional neurons. Taken together, the disclosure demonstrates the formation of functional neuro-vascular tissue with long-term culture capability.

It will be understood that various modifications may be made without departing from the spirit and scope of this disclosure. Accordingly, other embodiments are within the scope of the following claims.

What is claimed is:

1. A method of producing vascularized tissue in vitro comprising:
transforming a first population of pluripotent stem cell with a vector encoding one or more transcription factors that induce differentiation of the first population of pluripotent stem cell down a particular lineage path under the control of an inducible promoter to obtain parenchymal-inducible stem cells;
combining the parenchymal-inducible stem cells with a second population of pluripotent stem cells lacking the vector under suitable co-culture conditions; and
inducing the second population of pluripotent stem cells down a vascular committed lineage pathway and either before, during or after inducing vacularization inducing expression of the one or more transcription factors in the parenchymal-inducible stem cells to cause the parenchymal-inducible stem cells to become parenchymal cells, wherein a vascularized organoid tissue is obtained.

2. The method of claim 1, wherein the pluripotent stem cells are selected from embryonic stem cells, pluripotent stem cells, and induced pluripotent stem cells.

3. The method of claim 1, wherein the vector is a piggybac vector.

4. The method of claim 1, wherein the organoid tissue is a neuro-vascular tissue.

5. The method of claim 1, wherein the organoid tissue is a myo-vascular tissue.

6. The method of claim 1, wherein the parenchymal cell is selected from the group consisting of astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, and/or epididymal basal cells.

7. The method of claim 1, wherein the first population of pluripotent stem cells are layered in or on a first biocompatible matrix and the second population of pluripotent stem cells are layered in or on a second biocompatible matrix.

8. The method of claim 7, wherein the first and second biocompatible matrix comprise the same material.

9. The method of claim 7, wherein the first and second biocompatible matrix comprise different material compositions.

10. The method of claim 7, wherein the biocompatible matrix comprises a material selected from the group consisting of nylon (polyamides), dacron (polyesters), polystyrene, polypropylene, polyacrylates, polyvinyl compounds, polycarbonate (PVC), polytetrafluorethylene (PTFE, teflon), thermanox (TPX), nitrocellulose, cotton, polyglycolic acid (PGA), collagen, cat gut sutures, cellulose, and gelatin.

11. The method of claim 7, wherein the first and/or second biocompatible matrix are 3 dimensional porous materials.

12. The method of claim 1, wherein parenchymal-inducible stem cells are expanded in culture prior to combining with the second population of pluripotent stem cells.

13. A method of generating a vascular organoids comprising seeding a stem cell population comprising a vector containing one or more lineage committed transcription factors operably linked to an inducible promoter onto a vascularized organoid scaffold.

14. The method of claim 13, wherein the vascularized organoid scaffold is generated from stem cells.

15. An engineered vascular tissue produced by the method of claim 1, wherein the tissue comprises cells having an inducible promoter operably linked to a transcription factor.
